(19) United States
(12) Patent Application Publication
(10) Pub. No.: US 2013/0338635 A1
(43) Pub. Date: Dec. 19, 2013

(54) PRESSURE INDEPENDENT DELIVERY METHOD FOR PORTABLE INFUSION PUMP

(71) Applicant: Animas Corporation, West Chester, PA (US)

(72) Inventors: Sean O'Connor, West Chester, PA (US); Charles Hendrixson, West Chester, PA (US); Luis Jahn, (US)

(21) Appl. No.: 13/908,313

(22) Filed: Jun. 3, 2013

Related U.S. Application Data
(60) Provisional application No. 61/660,251, filed on Jun. 15, 2012.

(51) Int. Cl. A61M 5/168; A61M 5/142; A61M 5/172

(52) U.S. Cl. CPC A61M 5/16859; A61M 5/172; A61M 5/14244
USPC 604/506

(57) ABSTRACT

Described is method for maintaining proper drug delivery rate by monitoring atmospheric effects on a drug infusion device. The device may include one or more vents that permit the passage of gas between the exterior and interior of the device's housing. The device may also include one or more pressure and/or temperature sensors, the readings from which may be used to determine malfunctions in the venting of the device and/or changes in pressure that could cause the unintended over-delivery or under-delivery of medication.

### CROSS REFERENCE TO RELATED APPLICATIONS

This application relates to U.S. patent application Ser. No. 61/660,251, filed Jun. 15, 2012; all applications are herein incorporated by reference in their entireties.

### FIELD OF THE INVENTION

The present invention relates, in general, methods for delivering medications via a portable drug infusion device and, more particularly, to systems and methods for detecting and compensating for atmospheric effects in portable drug infusion devices.

### BACKGROUND OF THE INVENTION

The use of drug delivery devices for various types of drug therapy is becoming more common as the automated infusion of a drug may provide more reliable and more precise treatment to a patient.

Diabetes is a major health concern, as it can significantly impede on the freedom of action and lifestyle of persons afflicted with this disease. Typically, treatment of the more severe form of the condition, Type I (insulin-dependent) diabetes, requires one or more insulin injections per day, referred to as multiple daily injections. Insulin is required to control glucose or sugar in the blood, thereby preventing hyperglycemia that, if left uncorrected, can lead to ketosis. Additionally, improper administration of insulin therapy can result in hypoglycemic episodes, which can cause coma and death. Hyperglycemia in diabetics has been correlated with several long-term effects of diabetes, such as heart disease, atherosclerosis, blindness, stroke, hypertension, and kidney failure.

The value of frequent monitoring of blood glucose as a means to avoid or at least minimize the complications of Type I diabetes is well established. Patients with Type II (non-insulin-dependent) diabetes can also benefit from blood glucose monitoring in the control of their condition by way of diet and exercise. Thus, careful monitoring of blood glucose levels and the ability to accurately and conveniently infuse insulin into the body in a timely manner is a critical component in diabetes care and treatment.

To more effectively control diabetes in a manner that reduces the limitations imposed by this disease on the lifestyle of the affected person, various devices for facilitating blood glucose (BG) monitoring have been introduced. Typically, such devices, or meters, permit the patient to quickly, and with a minimal amount of physical discomfort, obtain a sample of their blood or interstitial fluid that is then analyzed by the meter. In most cases, the meter has a display screen that shows the BG reading for the patient. The patient may then dose theirselves with the appropriate amount, or bolus, of insulin. For many diabetics, this results in having to receive multiple daily injections of insulin. In many cases, these injections are self-administered.

Due to the debilitating effects that abnormal BG levels can have on patients, i.e., hyperglycemia, persons experiencing certain symptoms of diabetes may not be in a situation where they can safely and accurately self-administer a bolus of insulin. Moreover, persons with active lifestyles find it extremely inconvenient and imposing to have to use multiple daily injections of insulin to control their blood sugar levels, as this may interfere or prohibit their ability to engage in certain activities. For others with diabetes, multiple daily injections may simply not be the most effective means for controlling their BG levels. Thus, to further improve both accuracy and convenience for the patient, insulin infusion pumps have been developed.

Insulin pumps are generally devices that are worn on the patient's body, either above or below their clothing. Because the pumps are worn on the patient's body, a small and unobtrusive device is desirable. Some devices are waterproof, to allow the patient to be less inhibited in their daily activities by having to remove their drug infusion device while showering, bathing, or engaging in various activities that might subject their infusion device to moister, such as swimming. In such devices, it would be desirable to have a structure and method for verifying proper function of venting system within the device, since vents are typically passive devices that have no means for self-diagnostic checks to verify function has been compromised (i.e. intentional or unintentional obstruction of vent opening(s)). Further, it would be desirable to be able to alert the user of abnormal pressure differentials within their device that may cause erratic or unintentional drug delivery. Finally, it would be desirable for a drug infusion device to incorporate means for detecting the altitude at which the device is located, to avoid problems associated with air travel and sporting activities such as mountain climbing, skydiving, etc. that patients may wish to engage in without having to forego the use of their drug infusion device for concerns over erratic or unintentional drug delivery due to rapid pressure changes in and around the device.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates an exemplary embodiment of an drug infusion device having a pressure sensor for detecting pressure differentials between a sealed drug reservoir and the interior of the pump housing.

FIG. 2 illustrates an exemplary embodiment of a drug infusion device according to the present invention schematically.

FIG. 3 depicts an illustrative embodiment of a portable drug infusion device in cross-sectional view.

### DETAILED DESCRIPTION OF ILLUSTRATIVE EMBODIMENTS OF THE INVENTION

In an exemplary embodiment, the invention is directed to structures and methods for detecting pressure differentials between the compartment that houses the drug reservoir of a portable drug infusion pump and the external environment (atmosphere). Most portable insulin infusion pumps do not have a means for detecting air within the drug reservoir or line set. Such drug delivery systems operate under the premise that there is no air in the drug reservoir. Dosing controllers assumes that there a linear displacement of the drive mechanism that advances the cartridge plunger, thereby displacing a known volume of drug based on the constant area geometry of the cartridge barrel.

Typically, product labeling for these pump systems emphasizes the need to eliminate all air from the drug reservoir and line set prior to commencement of drug delivery. However, if air is present within the drug reservoir it will inherently lead to under infusion at some point during therapy. In addition, even when all precautions are taken to remove air from the drug reservoir, environmental factors such as changes in temperature and/or ambient pressure can cause air to come out of solution, which results in the formation of air bubbles in the drug reservoir or line set.

A further complication for portable infusion pump designers is that some portable infusion pumps are intended to be waterproof, to allow the patient wearing the device to maintain an active lifestyle and to allow the pump to be used during normal, daily activity, such as bathing. This is an attractive feature for people with lifestyles that benefit from continuous drug infusion (i.e. infusion of insulin for people with diabetes). Such devices must be designed with sealed enclosures/housings to prevent ingress of water. To avoid the development of pressure differentials between the external environment and the sealed compartment that houses the drug reservoir, most waterproof pumps incorporate hydrophobic vents that allow passage of air, but not fluids (within certain limitations of pressure differential).

Most portable drug infusion pump reservoirs developed from the most basic method of delivering medication—a standard syringe. Therefore, the reservoir is typically comprised of two major components; a cylindrical barrel, with a connector integrated into the distal end for attachment of an infusion line set, and a movable plunger with an elastomer seal. The plunger is inserted into the open proximal end of the barrel to form a closed volume. To deliver drug, a mechanically driven piston is advanced forward, which in turn advances the cartridge plunger forward, reducing the internal volume of the cartridge, thus displacing fluid. Typically, the piston (part of the durable device) is not mechanically interlocked with the cartridge plunger because there is no need to retract the plunger once the cartridge has been filled and subsequently installed in the pump.

If the pump piston is not interlocked with the cartridge plunger, there is a risk of unintentional delivery of drug if a positive pressure differential were to develop between the chamber that houses the reservoir and the external environment (location of infusion site). A positive pressure differential would impart a resultant force on the plunger which is directly proportional to the cross-sectional area of the drug reservoir's internal volume. If the resultant force exceeds the sustaining force of the cartridge plunger it will advance the plunger forward and thus deliver drug.

In one embodiment, the disclosed invention is a pump 100 with sealed housing that incorporates a differential pressure sensor 110 within the enclosure. The sensor 110 may be located in an exterior wall of the housing or in an interior wall that isolates the compartment that houses the drug reservoir 120 from the remainder of the internal volume of the pump. Those skilled in the art will recognize that this list in not exhaustive.

An infusion device as described may permit a method to verify proper function of the venting system. Vents are passive devices that typically have no means for self-diagnostic checks to verify function has been compromised (i.e. intentional or unintentional obstruction of vent opening(s)). The device may also alert the user (i.e. patient) of an increasing pressure differential prior to reaching a level that could result in unintentional delivery of drug. As well, if absolute pressure sensors are used (versus differential pressure sensor), the system could also double as an altimeter. This could be an attractive feature for end users with active life-styles (who were similarly attracted to a waterproof pump). Other benefits and advantages may exist, as those skilled in the art will recognize that detection of pressure differentials within the infusion device and/or the device being able to sense its altitude can provide for the implementation of a variety of features.

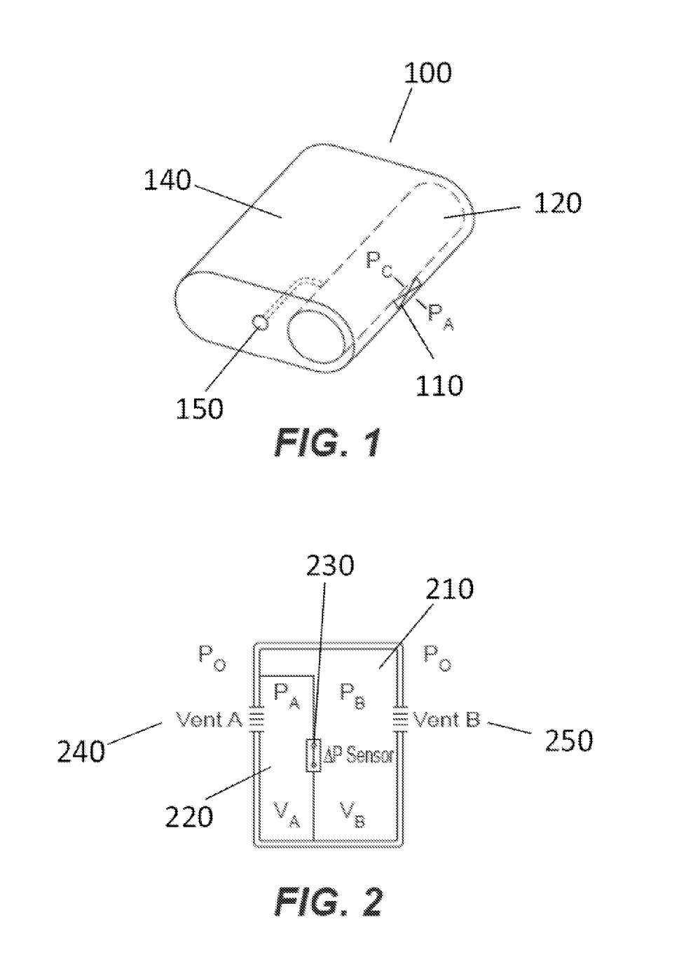

FIG. 1 illustrates a simplified view of a drug infusion device 100. The device may include a housing 110 with a sealed drug reservoir chamber 120 therein. The drug reservoir chamber may include a vent 150 to the atmosphere. In one embodiment of the invention, a pressure sensor 110 of the types well-known in the art is disposed in a manner that permits the measurement of the pressure differential between the drug reservoir chamber 120 and an adjacent compartment 140 of the interior of the drug housing. Although simplified for purposes of illustration, many drug infusion devices include multiple chambers within the housing and venting schemes to ensure pressure stabilization between them. Such schemes often include vents and membranes that permit gases to flow there through but inhibit the passage of moisture to maintain the waterproof or water resistant integrity of the device.

FIG. 2 shows the simplified interior of the device by way of an illustrative schematic. As shown, the simplified pump includes a housing 210 with multiple chambers 210, 220, each of which is separately vented to the atmosphere via vents 240, 250. Between the chambers a pressure sensor 230 is disposed. This design permits the detection of pressure differences between the chambers and the atmosphere and can allow for the creation of alerts or alarms to the user when certain preset-conditions are met.

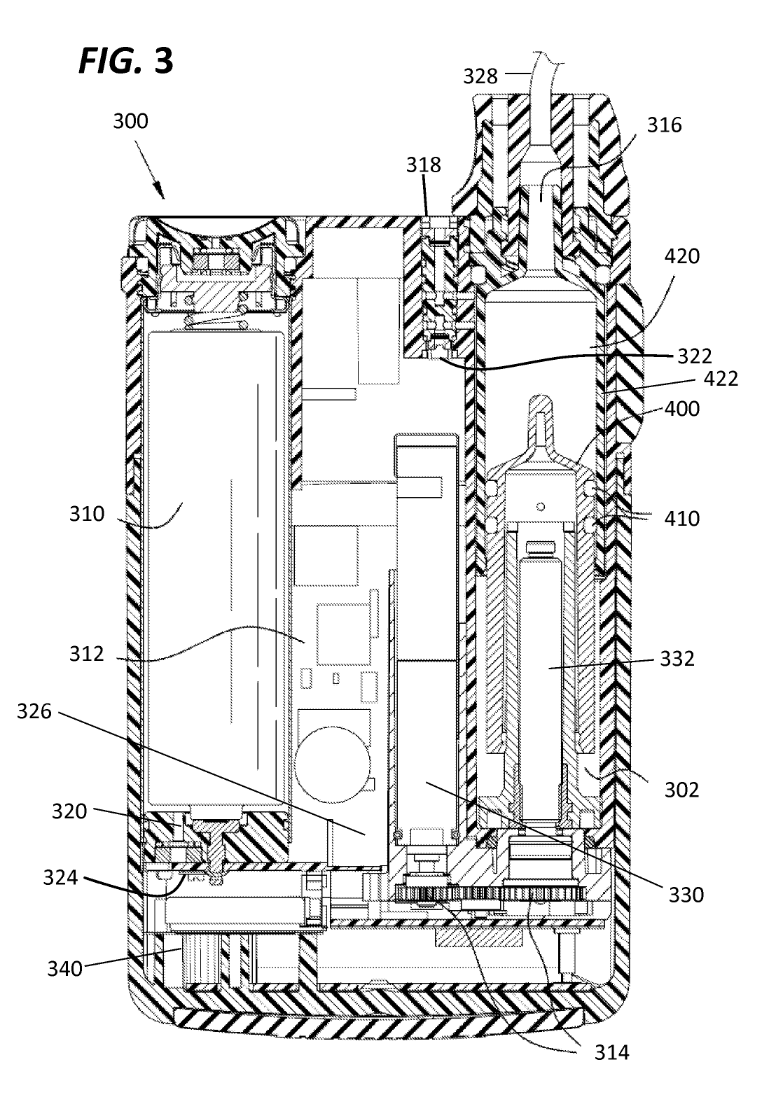

According to FIG. 3, a portable drug infusion device 300 might include a cavity for receiving and storing a drug cartridge 422 for holding a quantity of medication. O-rings 410 ensure a tight seal between the inner walls of the cartridge 400 and the plunger 400, thereby avoiding leakage of medication. The cavity 420 also includes an outlet port 316 to allow medication to flow into a lineset 328.

A motor 330, when actuated, turns gears 314 that extend a worm gear 332 into the plunger 302 to move the plunger 400, causing the interior volume of the cartridge 422 to decrease and fluid to be expelled via the outlet port 316 and into the lineset 328 (if attached). Power for the motor and other electrical components of the portable infusion device is provided by a battery that may be inserted via an opening provided for the battery cap 300 located into a battery compartment 310. A processor for controlling the motor, a display screen (not shown), a keypad interface, and various notification devices such as a vibration motor 340 can be located on a circuit board 312.

An exemplary method of the present invention relates to detection of air in the drug reservoir of a portable drug infusion device using two or more absolute pressure sensors. Data provided from the pressure sensors to the processor is then used to determine trending of the absolute pressure within the cartridge compartment. Use of absolute pressure sensors allows for monitoring of changes in altitude. One effect of decreasing atmospheric pressure (which occurs at increasing altitudes) is increased air in solution. This is due to reduced partial pressures. Increased air in solution, more specifically—increased air within the drug reservoir, could result in under-infusion, i.e. the patient receives a smaller amount of medication than intended. Another factor that could affect partial pressures is temperature. Therefore, a temperature sensor may be employed in conjunction with absolute pressure sensors.

Absolute pressure and temperature trending data could prove valuable when trying to understand certain unexpected outcomes. For example, in a continuous glucose monitoring (CGM) enabled insulin infusion pump system, insulin dosing regimens that were historically effective may not have the anticipated effect on reducing blood glucose values for reasons that are not readily apparent. Under such circumstances, one possible explanation would be under-delivery of insulin.

With cartridge compartment absolute pressure and temperature trend data available, algorithms could check for recent changes in pressure and/or temperature that could be indicative of increased amounts of air in the drug reservoir/cartridge. If recent changes in pressure and/or temperature were favorable for reduced partial pressures, an alarm could be triggered to prompt the user to check for the presence of air in their drug reservoir/cartridge.

Referring again to FIG. 3, the portable drug infusion device may include numerous vents to allow equilibration of the atmosphere with the interior of the device, such as a vent 320 between the battery compartment 310 and the interior of the device housing 300. Since the battery compartment 310 is typically not air-tight with respect to the interior of the housing 300, such a vent ensures that the internal pressure in the device is generally equal. Another vent 318 may be used to equilibrate the internal pressure of the device with the atmosphere, as well as the pressure within the cartridge cavity 302.

Absolute pressure sensors can be located a various portions of the interior of the housing. As shown in FIG. 3, absolute pressure sensors 322, 324 are located proximate to vents 318, 320. Optionally, a temperature sensor may also be located within the housing. In FIG. 3, a temperature sensor 326 is located on the circuit board 312.

Pressure sensors applicable for use in the various embodiments of the invention may include, but are not limited to, piezo-type sensors and MEMS sensors. They are generally preferable due to their small size and reliability. By monitoring the differential between the chambers within the housing, such as the sealed drug reservoir chamber and the external environment (ambient pressure), the device may be configured to trigger one or more of audible, tactible, or visual alarms. The user/patient may then be able to identify the source of the pressure differential and correct it or manually disconnect the drug infusion device to ensure that there is no unintended delivery of drug from what is typically a drug cartridge disposed in the sealed drug reservoir chamber. This method permits the preemptive detection of malfunctioning venting or another condition, rather than waiting until a degree of unintentional and possible harmful drug delivery has occurred.

Since some pressure sensors suitable for use according to the present invention are susceptible to damage or malfunction from such things as moisture and radiation (UV typically, but also IR), it is desirable for the pressure sensors to be mounted internal with respect to the exterior housing of the device. It has been found that the sensor may be particularly effective when positioned between two internal chambers of the device that are of different volume, as shown in FIG. 2, and both of the internal chambers 210, 220 are independently vented to ambient pressure.

Further, it is desirable that any differential pressure sensor used be capable of communicating with a microprocessor or other electronic device that controls and/or monitors the drug delivery device. This permits the user or manufacturer to program predetermined conditions into the microprocessor that will trigger an alarm when certain conditions are met—such a abnormal pressure differentials that indicate blockage of the housing vents, extremely low pressure such as might be encountered during airline depressurization, etc.

### Example 1

In an illustrative embodiment, the invention is a method for controlling the delivery rate of insulin to a patient and minimizing variations due to atmospheric effects such as altitude, pressure, and temperature changes. The method employs a portable, external pumping device, such as an insulin infusion pump similar to those sold by Animas Corporation of West Chester, Pa. under the trade names OneTouch® Ping®, Animas® 2020, and the like. The insulin pump has a housing that has two or more interior compartments, one of which may be used for a battery or other power source and another used to house circuit boards, etc.

Also within the housing is a cavity where a drug reservoir, such as a cartridge of insulin, is placed. Controlling the pump is a processor, a memory in communication with the processor, a display device. In this example, the display device is an organic light emitting diode display screen. The pump can also have one or more indicator devices controlled by the processor to create audible, visual, or tactile (e.g. vibratory) alarms to make the patient aware of certain conditions or in response to other pre-set parameters that may be stored in the device's memory or programmed into its operating software.

The patient or caregiver will typically interact with the device via a keypad, as is the instant case; however other user input devices such as touch screens, voice recognition, etc. may be used to communicate with the processor and interact with the operating software. The input device also allows the user to select between drug delivery programs that they may have created (or were created for them by their caregiver). The drug delivery program or programs are stored within the devices memory are may include basal and/or bolus delivery of their medication.

Greatly simplified, basal insulin is the background insulin that is normally supplied by the pancreas and is present 24 hours a day, whether or not the person eats. For most patients using insulin pump therapy, their pump will be running a basal insulin delivery program 24 hours each day. This makes the delivery rate of basal insulin more susceptible to changes in atmospheric conditions around the pump. Thus, when the patient goes in an airplane, hikes up a hill, or the weather changes significantly, there may be small variations in the basal insulin delivery rate actually produced by their drug infusion device due to changes in pressure, temperature, and/or altitude.

The present example employs absolute pressure sensors and temperature sensors in drug infusion device to monitor changes in temperature, pressure, and altitude. By, in this instance, deploying absolute pressure sensors proximate to the interior inlet of each vent in the insulin pump, as well as a temperature sensor, atmospheric data can be monitored and sent to the processor. The processor may then employ algorithms to determine a correction factor to the basal insulin delivery rate to ensure that the amount actually being delivered is the amount expected by the patient. The processor applies the atmospheric correction factor to the drug delivery program (such as basal insulin delivery program running on the processor or stored in memory). The processor, by controlling the speed and movement of the motor drive, can alter (increase or decrease) the drug delivery rate.

It will be recognized that equivalent structures may be substituted for the structures illustrated and described herein and that the described embodiment of the invention is not the only structure, which may be employed to implement the claimed invention. In addition, it should be understood that every structure described above has a function and such structure can be referred to as a means for performing that function. While embodiments of the present invention have been shown and described herein, it will be obvious to those skilled in the art that such embodiments are provided by way of example only. Numerous variations, changes, and substitutions will now occur to those skilled in the art without departing from the invention.

It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for regulating drug delivery, comprising:
providing a portable, external pumping device comprising a housing having two or more interior compartments therein, a cavity within the housing for receiving a drug reservoir, a processor, a memory in communication with the processor, a display device controlled by the processor, one or more indicator devices controlled by the processor, a user input device in communication with the processor, a drug delivery program stored within the memory, a motor drive controlled by the processor configured to drive a plunger and expel fluid from the drug reservoir, at least one external vent permitting passage of gas between the interior and exterior of the housing, at least one internal vent permitting passage of gas between the two or more interior compartments, at least one temperature sensor located in the interior of the housing, and two or more absolute pressure sensors;
sensing a temperature within the housing;
sensing a first pressure;
sensing a second pressure;
determining based on the first pressure, the second pressure, and the temperature an atmospheric correction factor;
applying the atmospheric correction factor;
modifying the drug delivery program; and
controlling the motor drive to deliver insulin in accordance with the modified drug delivery program.
